(19) United States
(12) Patent Application Publication
(10) Pub. No.: US 2017/0340445 A1
(43) Pub. Date: Nov. 30, 2017

(54) Ceramic Spacer for the Two-Sided Replacement of Implants in Shoulder, Knee and Hip as a Result of Infections

(71) Applicant: CeramTec GmbH, Plochingen (DE)

(72) Inventors: Andreas REMPP, Stuttgart (DE); Ralph AUTENRIETH, Esslingen (DE); Alfons KELNBERGER, Röthenbach (DE); Roman PREUSS, Kirchheim unter Teck (DE); Heinrich WECKER, Eckental (DE); Tobias WEISS, Ebersbach (DE); Javad PARVIZI, Gladwyne, PA (US)

(73) Assignee: CeramTec GmbH, Plochingen (DE)

(21) Appl. No.: 15/532,914

(22) PCT Filed: Dec. 3, 2015
(86) PCT No.: PCT/EP2015/078460

§ 371 Date: Jun. 2, 2017

(30) Foreign Application Priority Data
Dec. 5, 2014 (DE) 10 2014 225 026.9

(51) Int. Cl. A61F 2/30; A61F 2/38; A61F 2/40; A61F 2/36

(52) U.S. Cl. CPC A61F 2/30; A61F 2/3662; A61F 2/3859; A61F 2/389; A61F 2/4014; A61F 2/4059; A61F 2/36; A61F 2002/3619; A61F 2002/3615; A61F 2002/30677; A61F 2002/30672; A61F 2002/30649; A61F 2002/30624; A61F 2002/305; A61F 2002/30064; A61F 2002/3654; A61F 2002/4022; A61F 2002/4025; A61F 2002/4055; A61F 2310/00011; A61F 2310/00353; A61F 2310/00179

(57) ABSTRACT

A spacer comprised of individual components as joint replacement in the arthroplasty of shoulder, knee, and hip for the temporary surgical care of infections in the joint. The problems addressed by the invention are those of avoiding an adhesion of the bone to the spacer and a release of wear products, achieving that the patient does substantially not experience any mobility restriction, and keeping the wear and tear of the spacer as little as possible. These problems are solved in that at least one component of the spacer is made of a ceramic material, of a plurality of ceramic materials, or of a non-ceramic material having a ceramic coating.

The invention relates to a spacer consisting of individual components as a joint replacement in the arthroplasty of the shoulder, knee and hip for the temporary surgical care of infections in the joint.

Spacers are known in the field of joint arthroplasty and are inserted temporarily to support the healing of bacterial infections resulting from endoprosthetic treatment and to permit minimal mechanical functionality of the joint apparatus during the healing phase.

The healing is supported by the localized and, if possible, controlled administration of antibiotic or antibacterial substances. A minimal functionality of the joint is achieved when the spacer has at least the following design elements:
distally, a pedicle or stem, which is comparable to the pedicle or stem of an artificial hip joint that braces the spacer in the medullary canal;
proximally, a ball segment or a ball head, which, to a limited extent (primarily with regard to the number of changes in load and movement), is capable of articulating in the hemisphere of the acetabular cavity, which remains after the artificial hip socket is removed.

The service life/use of the spacer ends once the infection has healed after several weeks; the spacer is removed and a new endoprosthesis, optionally a revision endoprosthesis, is inserted.

Spacer systems have heretofore been based primarily on PMMA bone cements that have been mixed or filled with antibiotic substances, mostly gentamicin and/or vancomycin, and either delivered as prefabricated implants or produced using provided material mixtures and molds during the surgical operation.

Numerous fundamental disadvantages or restrictions arise in the process—depending upon the system employed—which have negative effects on the patient:
As retention time within the patient increases, the risk of the bone adhering to the spacer rises, which complicates the removal of the merely temporary implant and results in undesirable bone loss during the removal. This risk is compounded by the surface roughness specific to the material and shape and by the chemical and biological properties of the materials used.
The low wear resistance of the materials that are used combined with tribological stress places extreme constraints on the patient's mobility. The wear products that are released—cement particles as well as ceramic particles with which the cements are often filled to strengthen them—can provoke negative biological reactions and increase the risk of wear by so-called third-party wear over the service life of the installed endoprosthesis.
The low mechanical strength of the temporary spacer compared to regular prostheses forces the patient to severely restrict the load (height of the load and number of load cycles) and thus his or her own mobility in order to prevent the spacer from failing as a result of a rupture.
Owing to the crude design of the cement spacer, it is usually possible to produce or provide only very imprecise friction pairings, which results in increased wear and insufficient guidance and stability of the joint.

The invention addresses the problem of improving a spacer according to the preamble of claim 1 so as to prevent the adhesion of the bone to the spacer and the release of wear products. Moreover, the patient should experience practically no restriction of mobility, and the wear of the spacer should be as low as possible, as this can result in increased wear and insufficient guidance and stability of the joint. The present invention eliminates these disadvantages and produces several further advantages, which will be described below.

The stated problem is solved by a spacer according to claim 1.

Because the spacer consists of at least one component composed of a ceramic substance or multiple ceramic substances or of a non-ceramic substance with a ceramic coating, the adhesion of the bone to the spacer and the release of wear products are prevented. Moreover, the patient experiences practically no restriction of mobility, and the wear of the spacer is as low as possible, and there is thus no increased wear or insufficient guidance and stability of the joint.

Preferably, all exposed surfaces of the at least one ceramic component that are in contact with biological tissue are polished or processed such that their surface roughness Ra is less than 0.35 μm. The adhesion of the bone is thereby prevented.

To promote the healing process, at least some regions of the at least one ceramic component have a coating or multiple coatings with one or more antibiotic agents.

In one embodiment, the spacer consists of a ceramic stem with a projecting retainer element, a metallic sleeve being attached to the retainer element and a ceramic ball head being anchored to the sleeve. The spacer thus consists of the same components as a traditional prosthesis.

The coupling between the stem and sleeve and between the sleeve and ball head is preferably established by a conical clamping connection, or the coupling between the stem and sleeve is established as a clamping connection based on frustum geometries or in a form-fitting manner based on a square geometry, e.g. in order to promote the shaping of the stem by the single-axis dry pressing of ceramic powder, or the coupling is established in a firmly bonded or form-fitting way by using bone cement.

The sleeve can also be clamped to the retainer element by one or more stops.

To prevent the stem from rotating in the bone, the section of the stem that engages in the bone has a rectangular or oval shape in cross-section.

For a joint replacement in the arthroplasty of the knee, the spacer preferably consists only of two components, namely a femur component composed of bone cement or a ceramic substance and a tibia component composed of a ceramic substance, said femur component having two parallel sliding surfaces that are mounted on two parallel curved regions on the tibia component.

The femur component is preferably closed between the sliding surfaces to improve stability.

To prevent rotation, the tibia component has a pin on the lower side opposite the curved regions, which protrudes at a right angle and which is non-circular in cross-section and which preferably has an angular or oval cross-section.

The invention thus describes a spacer in one or multiple parts for the temporary surgical treatment of infections in arthroplasty, consisting of one or more ceramic substances in combination with interface modules composed of plastic or metal, optionally also partly or entirely coated with an antibiotic agent or antibacterial materials.

Advantageously:
all exposed surfaces (surfaces in contact with biological tissue) of the spacer are polished or otherwise processed with the objective of reducing the surface roughness Ra in order to reduce adhesion and the development of biofilms by bacteria;
the coating releases one or more antibiotic agents while in the human body, e.g. in the form of a gentamicin-containing palmitate layer—alternatively, the coating can also be embodied in the form of antibiotic metals, such as silver or copper, which prevent a bacterial colonization without necessarily releasing agents;
the release kinetics of the agent can be adapted to the patient's needs, e.g. by means of multiple layers with different concentrations of the agent and/or layer thicknesses, when the release occurs in timed relation to the penetration of the layers by bodily fluids or by the degradation of the layers (biological degradation) and the release of the agent thus occurs from the outside in.

Suitable ceramic materials:
high-purity alumina, in particular the CeramTec material Biolox Forte;
zirconia-toughened alumina (ZTA), in particular the CeramTec material Biolox Delta;
yttrium-stabilized zirconia (3Y-TZP), in particular the CeramTec material MZ111;
cerium-stabilized zirconia (Ce-TZP), in which the tetragonal phase of the zirconia is stabilized by ceria;
yttrium-stabilized zirconia with secondary phases that contain, for example, strontium aluminates as dispersoids (SFIYTZ) and achieves the highest values for the parameter fracture toughness.
Of course, all variants of these agent classes are suitable for the claimed component.
Non-oxide ceramic agents, such as silicon nitride Si3N4, can also be considered as the ceramic materials for the present invention.

Embodiments for use in hip and shoulder arthroplasty—advantageously:
the system has a modular structure and consists of a stem with a mounted adapter sleeve and, in turn, a mounted ball head or ball segment head; see the drawings;
the ceramic ball head basically resembles the conventional ball heads of hip arthroplasty, but satisfies lower requirements regarding geometric tolerances and surface finish related to its use;
the diameter of the ceramic ball head 4 is in the range between 48 and 60 mm, since it articulates against the cavity that remains after the removal of the metal socket in the acetabulum or against the natural acetabulum if there is an infection in a hemi-endoprosthesis—gradations in 6 mm steps are economically sensible, and other gradations are of course likewise possible;
the functional zone of the articulating sphere of the ball head can be smaller than is conventional in a hip endoprosthesis (ball segment);
the coupling between the stem and sleeve and between the sleeve and ball head is preferably established by a conical clamping connection—alternatively, the coupling between the stem and sleeve can also be established as a clamping connection based on frustum geometries or in a form-fitting manner based on a square geometry, e.g. in order to promote the shaping of the stem by the single-axis dry pressing of ceramic powder—alternatively, the coupling is established in a firmly bonded or form-fitting way by using bone cement (optionally including an antibiotic agent such as gentamicin) that is blended during surgery;
in the case of a conical clamping connection, the coupling is established by means of larger cone diameters than in conventional hip prostheses so as to produce larger stem neck diameters, which reduces the bending stresses in the ceramic stem neck compared with metal stems;
the sleeve consists of an established implant alloy such as TiAl6V4, CoCrMo, stainless steel or pure titanium—alternatively, the sleeve can also consist of a biocompatible polymer such as PE, XPE, vitamin E XPE, PEEK, PEKK or PEMA;
the embodiments belong to a system of multiple sleeves with different inner and/or outer diameters, but only one of which can be implanted at a time (cannot be combined), and which thereby permit the position of the ball head along the stem neck axis to be varied (so-called “neck jump” or “neck length”);
sleeves composed of polymer materials do not have a purely force-fitting connection (e.g. conical clamping connection), but instead also have one or more stops, which become active when a predefined state of tension is reached in the clamping connection as a result of the accompanying relative displacement;
the ceramic stems have the basic shapes of known implant systems; the spacer is even more space-saving when selected on the basis of the extracted, infected system (retaining the existing bone bed) and/or the new or revision system that will be implanted (retaining the bone bed that has been created for the remainder of the procedure)—economic considerations can also include offering a reduced variety of shapes that are oriented to established, market-leading systems—alternatively, a uniform geometry could be possible, which would permit large quantities with little variety;
the ceramic stem has a collar on the proximal part, which provides additional support and thereby contributes to preventing both the countersinking of the stem and adhesion.

Alternatively, a dense ceramic coating can be applied to an established hip stem made of a metallic substance, and so the stem is then sufficiently similar to a full ceramic stem in terms of its surface and its chemical, physical and biological properties to reduce osseointegration and bacterial colonization.

The invention will be further explained below on the basis of drawings.

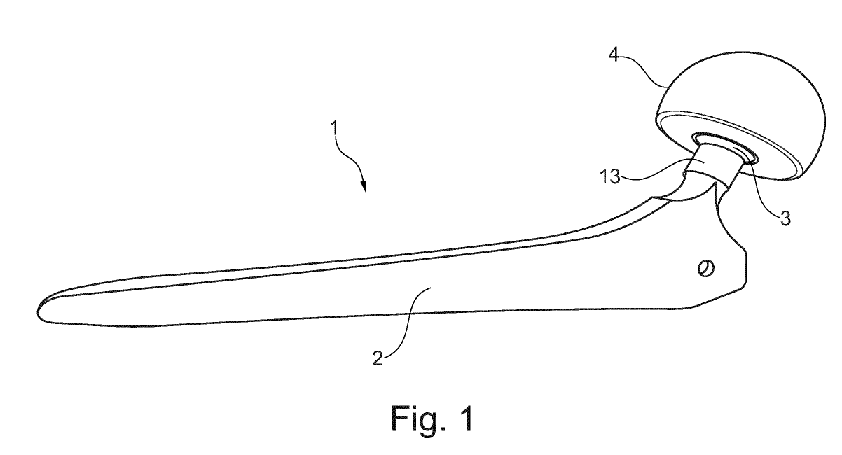

FIG. 1 shows a claimed spacer 1, consisting of a stem 2 with a projecting retainer element 13 for a sleeve 3 and a head 4. The stem 2 and the head 4 consist of a ceramic material. In the present embodiment, the sleeve 3 consists of a metal. The cross-section has a rectangular or oval shape so that the stem 2 can be implanted such that it does not rotate in the bone.

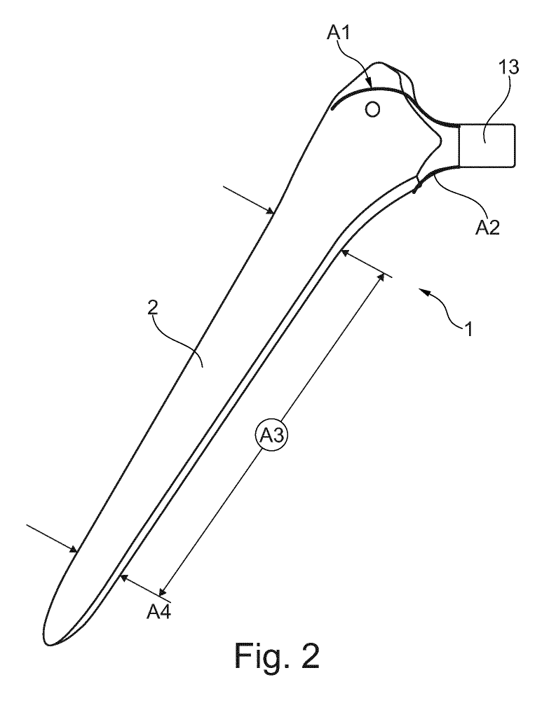

FIG. 2 shows the stem 2 alone. It has an anatomical design at the position indicated A1. The stem has large radii at indicated position A2. In region A3, there are only smooth transitions in the cross-section. The stem has a maximum cross-section of 14*14 mm in region A4.

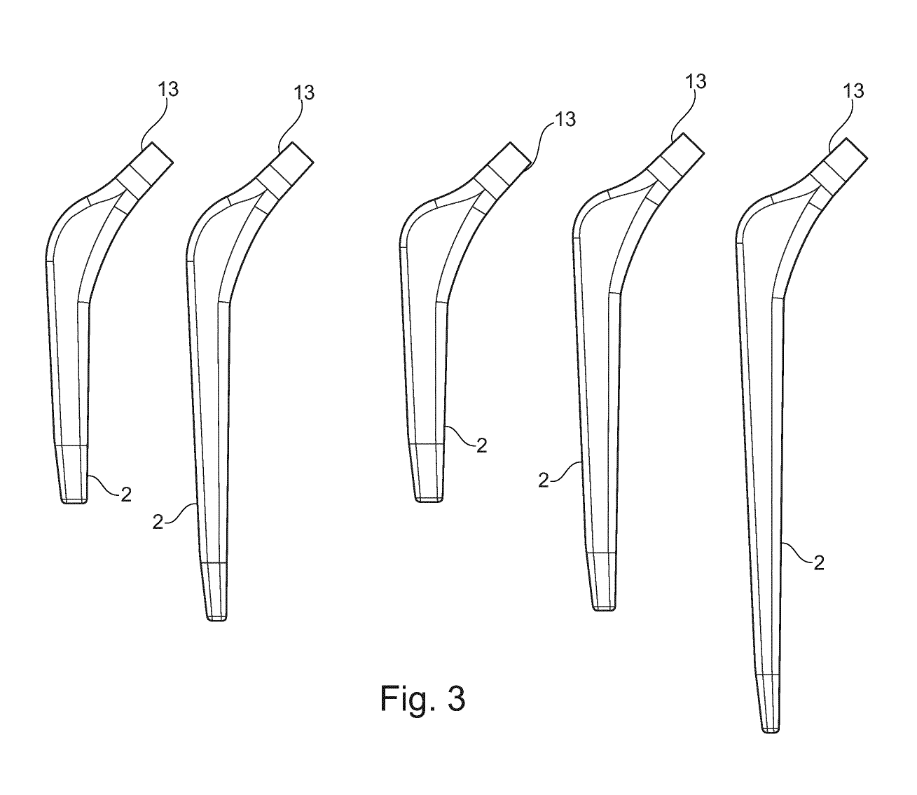

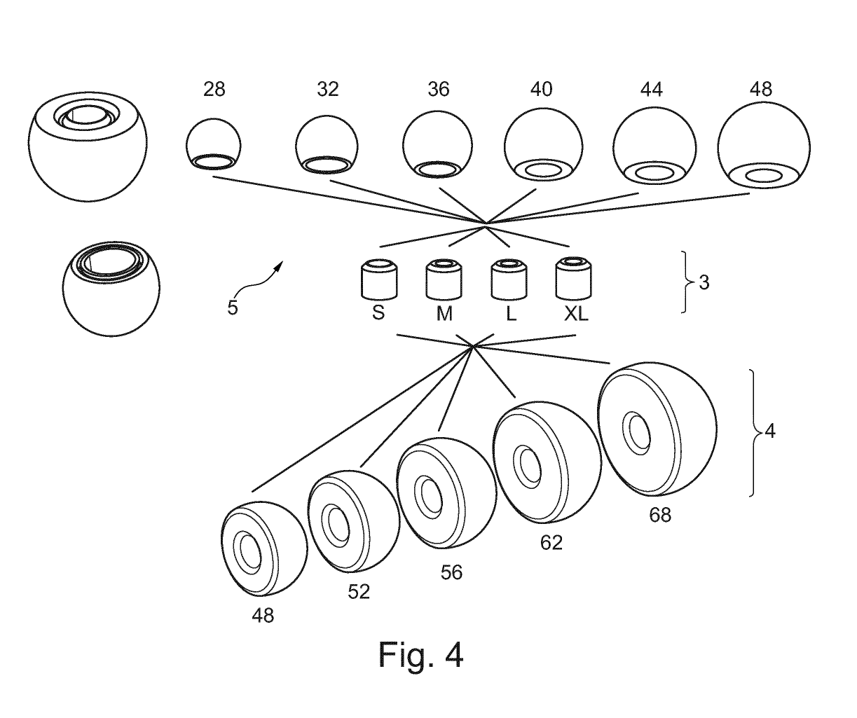

FIG. 3 shows various sizes of the stem 2. FIG. 4 shows conventional ball heads 5 compared with the ball heads 4 for the spacers including the sleeves 3. The ball heads 4 are larger than the conventional ball heads 5 in the hip prosthesis.

The surface roughness Ra of the stem 2 after sintering is 0.25 to 0.35. The ball head 4 of the spacer 1 has a surface roughness of preferably 0.1 μm.

Embodiment for use in arthroplasty of the knee:

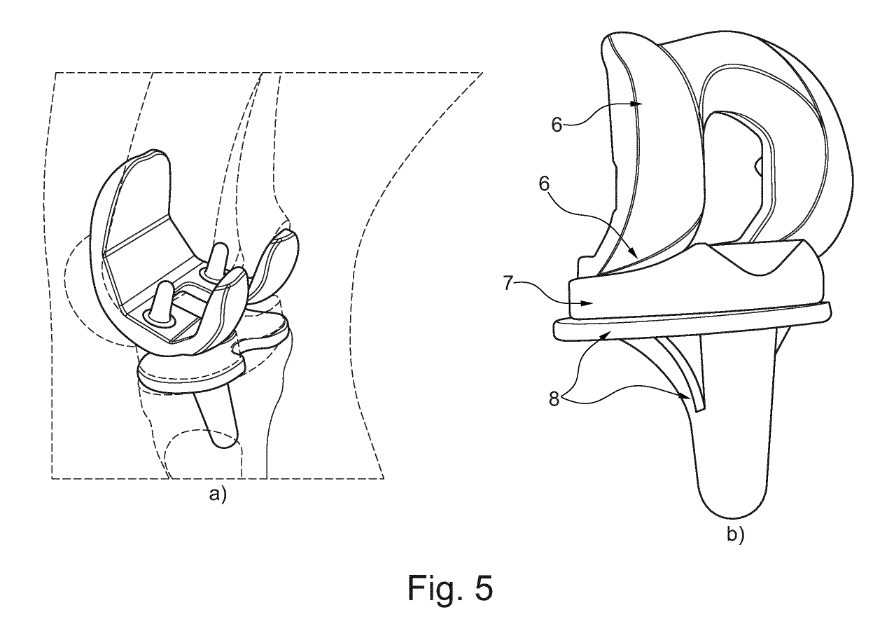

FIGS. 5a and 5b show the prior art of knee joint prostheses. This prosthesis consists of a ceramic femur component 6, a PE component 7 and a ceramic tibia component 8. FIG. 5a shows the prosthesis implanted and FIG. 5b shows it not yet implanted.

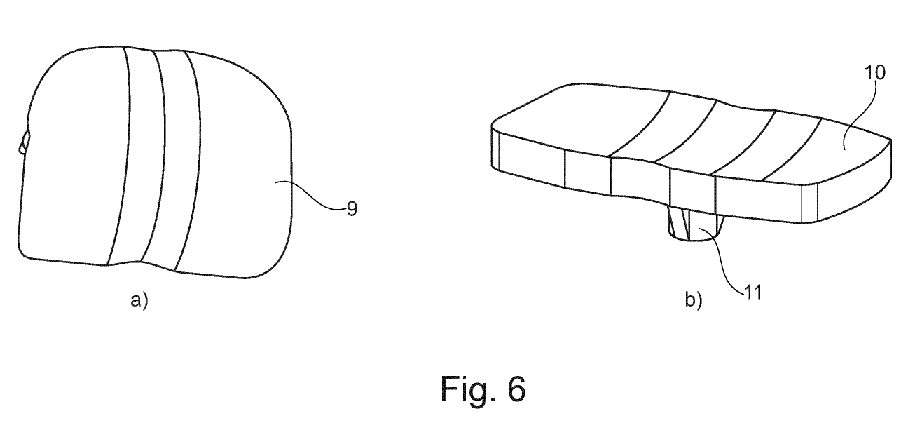

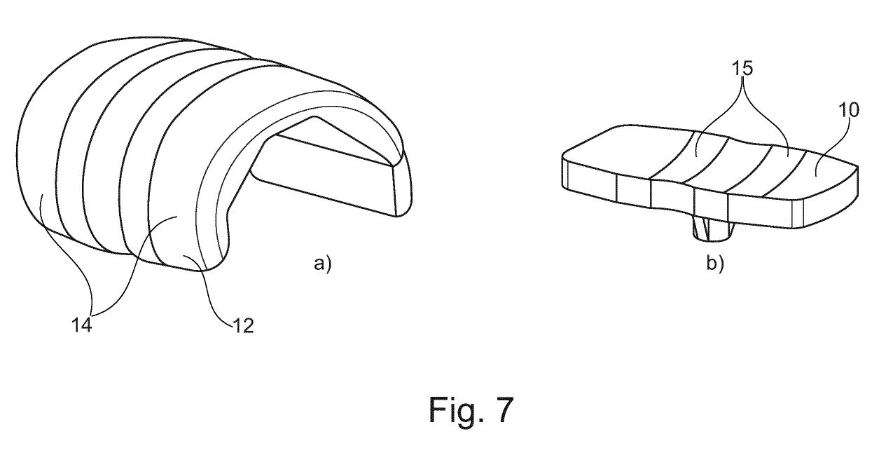

Claimed spacers for the knee are depicted in FIGS. 6 and 7.

FIG. 6a shows a femur component 9 that consists of bone cement and is mounted on a claimed tibia component 10 composed of a ceramic material. The tibia component 10 consists substantially of a rectangular plateau composed of a ceramic material with curved regions 15, on which the femur component 9 is mounted by its sliding surfaces 14. The tibia component 10 has a pin 11 on its lower side, said pin being non-circular, e.g. with an angular or oval cross-section, to secure it against rotation. Both the femur component 9 and the tibia component 10 are anchored in the bone with bone cement.

An alternative spacer is shown in FIGS. 7a and 7b. Like the tibia component 10, the femur component 12 in this embodiment consists of a ceramic material. The tibia component 10 is identical in configuration to the tibia component 10 according to FIG. 6b.

Advantageously:
the femur component 9, 12 of the spacer has simplified geometry and is modelled on existing spacers composed of PMMA materials—alternatively, a universal geometry can be implemented, which represents the average of the inner contour of the most common implant systems on the market or the minimal geometry (minimum material condition) of an overlap between these systems;
the tibia component 10 is executed in the form of a simplified plateau of ceramic, where the joint space is created by inserting one or more stackable spacer plates;
the spacer plates consist of ceramic material or a low-abrasion polymer, e.g. PE, XPE, vitamin E XPE, wherein the polymer could additionally be filled with one or more antibiotic agents, which are released in vivo.

Production of the ceramic components:

The articulating components can be produced by the known production methods, such as uniaxial or isostatic dry pressing, shape cutting, single or multi-stage sintering (hot isostatic pressing is possible but may not be necessary), grinding and polishing in the sintered state, wherein processing by grinding and polishing can be reduced or sub-steps omitted owing to lower requirements regarding tolerances and surface finishes.

The articulating components could likewise be shaped by ceramic injection molding (CIM or LIM).

For applications in hip or shoulder arthroplasty, the following methods for producing the stem 2 are possible:
slip casting
dry pressing
wet pressing
true net-shape CIP (cold isostatic pressing)
CIM (ceramic injection molding)
gel casting
LIM (low pressure injection molding)

After sintering, the surface of the ceramic stem is mechanically reprocessed, optionally by grinding and/or vibratory grinding and/or polishing, until the smoothest possible surface is obtained, which undergoes little or no osseointegration into the bone and prevents bacterial colonization but which permits sufficient adherence of the coating, if needed.

For applications in hip or shoulder arthroplasty, the metallic sleeve 3 is advantageously produced in a cost-saving manner by deep-drawing from sheet metal blanks.

1. A spacer having individual components as a joint replacement in the arthroplasty of the shoulder, knee and hip for the temporary surgical care of infections in the joint, characterized in that the spacer comprises at least one component that is composed of one or more ceramic substances or of a non-ceramic substance with a ceramic coating.

2. The spacer according to claim 1, wherein all exposed surfaces of the at least one ceramic component that are in contact with biological tissue are polished or processed such that their surface roughness Ra is less than 0.35 μm.

3. The spacer according to claim 1, wherein at least some regions of the at least one ceramic component have a coating or multiple coatings with one or more antibiotic agents.

4. The spacer according to claim 1 for a joint replacement in the arthroplasty of the hip and the shoulder, wherein the spacer consists of a ceramic stem with a projecting retainer element, a metallic sleeve being attached to said retainer element, and a ceramic ball head being anchored to the sleeve.

5. A spacer according to claim 4, wherein the coupling between the stem and sleeve and between the sleeve and ball head is preferably established by a conical clamping connection, or the coupling between the stem and sleeve is established as a clamping connection based on frustum geometries or in a form-fitting manner based on a square geometry, e.g. in order to promote the shaping of the stem by the single-axis dry pressing of ceramic powder, or the coupling is established in a firmly bonded or form-fitting way by using bone cement.

6. The spacer according to claim 4, wherein the sleeve can also be clamped to the retainer element by one or more stops.

7. The spacer according to claim 1, wherein the section of the stem that engages in the bone has a rectangular or oval shape in cross-section.

8. The spacer according to claim 1 for a joint replacement in the arthroplasty of the knee, wherein the spacer consists only of two components, namely a femur component composed of bone cement or a ceramic substance and a tibia component composed of a ceramic substance, said femur component having two parallel sliding surfaces that are mounted on two parallel curved regions on the tibia component.

9. The spacer according to claim 8, wherein the femur component is closed between the sliding surfaces.

10. The spacer according to claim 8, wherein the tibia component has a pin on the lower side opposite the curved regions, which protrudes at a right angle and which is non-circular in cross-section and which preferably has an angular or oval cross-section.
